(19) United States
(12) Patent Application Publication
(10) Pub. No.: US 2026/0207959 A1
(43) Pub. Date: Jul. 23, 2026

(54) CLOSED-LOOP, NON-INVASIVE BRAIN STIMULATION SYSTEM AND METHOD RELATING THERETO

(71) Applicant: RESONAIT MEDICAL TECHNOLOGIES PTY LTD, Terrigal, New South Wales (AU)

(72) Inventor: Cameron HIGGINS, Terrigal, New South Wales (AU)

(21) Appl. No.: 19/141,894

(22) PCT Filed: Dec. 21, 2023
(86) PCT No.: PCT/AU2023/051354

§ 371 Date: Jun. 20, 2025

(30) Foreign Application Priority Data
Dec. 21, 2022 (AU) 2022903932
Dec. 21, 2022 (GB) 2219341.1

(51) Int. Cl. A61N 2/00; A61N 2/02

(52) U.S. Cl. CPC A61N 2/006; A61N 2/02

(57) ABSTRACT

Disclosed herein are a brain stimulation method and system for use on a mammal. The system includes: an electroencephalogram (EEG) headset (110) having sensors for detecting an electrical signal of a patient, and an EEG processing device (120) for determining a stimulation protocol based on that electrical signal. The EEG processing device (120) includes: a brain state inference module (124) for determining an inferred brain state of the patient; and a learning module (126) for learning the stimulation protocol based on the detected electrical signal and the inferred brain state. The system also includes a neuromodulator (130) for delivering stimuli to the patient based on the stimulation protocol.

### RELATED APPLICATIONS

This application is related to Australian Provisional Patent Application No. 2022903932 titled “Closed-loop, non-invasive brain stimulation system and method relating thereto” and filed 21 Dec. 2022, United Kingdom Patent Application No. 2219341.1 filed 21 Dec. 2022, and Australian Provisional Patent Application No. 2023904187 filed 21 Dec. 2023, the entire content of each of which is incorporated by reference as if fully set forth herein.

### TECHNICAL FIELD

The present disclosure relates to a system and associated method to elicit or inhibit target brain states in mammals. In particular, the present disclosure relates to a closed-loop, non-invasive brain stimulation system and related method for use in association with mammals, particularly humans, to elicit or inhibit target brain states.

### BACKGROUND

Brain stimulation is an emerging form of medical therapy for the treatment of mental health and neurological disorders. By directly stimulating neural tissue in specific cortical sites, or indirectly stimulating the brain by controlling sensory input with a certain temporal protocol, it is possible to induce clinical effects that can alleviate the symptoms of chronic mental health and neurological disorders.

There are a number of different brain stimulation techniques presently known, including Transcranial Magnetic Stimulation (TMS), Transcranial Electrical Stimulation (TES), Closed Loop Auditory Stimulation (CLAS), Audio-Visual Stimulation (AVS) and Transcranial Ultrasound Stimulation (TUS). However, there is often no clear understanding of how each of the known brain stimulation techniques translates to effects on long term neural activity and subsequently to clinical outcomes, including therapeutic outcomes.

Desired changes in brain states that are sought to be elicited by brain stimulation therapy to achieve a therapeutic goal for a particular patient cohort may be referred to herein as “therapeutic outcome targets”. It is presently easier for clinicians to specify therapeutic outcome targets than to specify the stimulation parameters that may elicit therapeutic outcome targets.

Current approaches to brain stimulation by neuroscientists and engineers typically forward engineer the stimulation parameters applicable to a subject. That is, the neuroscientists and engineers typically set stimulation parameters by some procedure at the start of a session on the basis of strong assumptions about how stimulation will modulate a patient's brain activity. Those stimulation parameters then typically remain fixed for the duration of the stimulation of the participant, regardless of whether the stimulation is actually achieving its goal.

Determining the stimulation parameters that will maximally elicit changes in default mode brain network activity, for example, is a challenging task. The default mode brain network is characterised by activity distributed across nodes in the frontal medial, medial and lateral parietal, and medial and lateral temporal cortices of the brain. The maximal effect may be achieved by stimulating in any one of these nodes; or by stimulating in a neural tract that connects two or more such nodes; by stimulating one such node at a specific frequency or amplitude; or by stimulating an entirely different brain network that is strongly anticorrelated with the default mode network, such as the dorsal attention network.

Further complicating matters is that neurological disorders and mental health conditions are very idiosyncratic in their expression over different individuals, such that the ideal brain stimulation protocol for two different patients can potentially be extremely different.

For example, the spatial positioning of TMS coils is sometimes customised by neuro-navigation models built from a subject's pre-recorded Magnetic Resonance Image (MRI), to ensure stimulation is delivered to a specific cortical site. However, there is no monitoring of the neural response of a patient when that site is stimulated to determine if that cortical site is, in fact, an appropriate target, and if such stimulation is achieving or undermining the underlying clinical objectives for that particular individual. Similarly, common methods for titrating the strength of the stimulation applied to an individual use proxy measures that do not typically have any direct relationship to clinical outcomes, including therapeutic outcome targets.

Thus, a need exists to provide a method and system to tailor brain stimulation protocols to each individual patient, on the basis of the unique response of each respective patient to stimulation, and predefined therapeutic outcome target(s).

### SUMMARY

The present disclosure relates to a system and associated method to elicit target brain states in mammals. In particular, the present disclosure relates to a closed loop, non-invasive brain stimulation system and related method for use in association with mammals, particularly humans.

A first aspect of the present disclosure provides a brain stimulation system for use on a mammal comprising:
an electroencephalogram (EEG) headset having a plurality of EEG sensors for detecting an electrical signal of a patient, each sensor corresponding to a channel of the signal;
a computer-implemented EEG processing device for determining a stimulation protocol based on said detected electrical signal, wherein said EEG processing device includes:
   a brain state inference module for determining an inferred brain state of the patient for a given time based on said detected electrical signal and a set of predefined brain states, wherein each predefined brain state is associated with a set of brain state parameters including corresponding brain network patterns and brain network dynamics, said brain state inference module determining a likelihood of the patient being in each of said predefined brain states based on (i) correspondence of said detected electrical signal with said set of brain network patterns associated with each of said predefined brain states, and (ii) said brain network dynamics, wherein said inferred brain state has the highest overall likelihood for said given time, and
   an artificial-intelligence implemented learning module for learning said stimulation protocol based on the detected electrical signal and a predefined therapeutic outcome target; and
   a neuromodulator for delivering stimuli to said patient based on said stimulation protocol.

A second aspect of the present disclosure provides a method of stimulating a brain of a mammal comprising the steps of:
utilising an electroencephalogram (EEG) headset to detect an electrical signal of a patient, said EEG headset having a plurality of EEG sensors wherein each sensor corresponds to a channel of the signal;
determining a stimulation protocol based on said detected electrical signal by utilising a computer-implemented EEG processing device, wherein said EEG processing device includes:
   a storage medium for storing a set of predefined brain states, each brain state being associated with a set of brain state parameters including corresponding brain network patterns and brain network dynamics;
   a brain state inference module for determining an inferred brain state of the patient for a given time based on said detected electrical signal and said set of predefined brain states, said brain state inference module determining a likelihood of the patient being in each of said predefined brain states based on: (i) correspondence of said detected electrical signal with said set of brain network patterns associated with each of said predefined brain states, and (ii) said brain network dynamics, wherein said inferred brain state has the highest likelihood for said given time, and
   an artificial-intelligence implemented learning module for learning said stimulation protocol based on the detected electrical signal and said inferred brain state; and
delivering stimuli to said patient, via a neuromodulator, based on said stimulation protocol.

A third aspect of the present disclosure provides a brain stimulation system for use on a mammal comprising:
a neurophysiological sensing device having a plurality of sensors for detecting a neurophysiological signal of a patient, each sensor corresponding to a channel of the signal;
a computer-implemented processing device for determining a stimulation protocol based on said detected neurophysiological signal, wherein said processing device includes:
   a brain state inference module for determining an inferred brain state of the patient for a given time based on said detected electrical signal and a set of predefined brain states, wherein each predefined brain state is associated with a set of brain state parameters including corresponding brain network patterns and brain network dynamics, said brain state inference module determining a likelihood of the patient being in each of said predefined brain states based on: (i) correspondence of said detected electrical signal with said set of brain network patterns associated with each of said predefined brain states, and (ii) said brain network dynamics, wherein said inferred brain state has the highest likelihood for said given time, and
   an artificial-intelligence implemented learning module for learning said stimulation protocol based on the detected electrical signal and said inferred brain state; and
a neuromodulator for delivering stimuli to said patient based on said stimulation protocol.

According to another aspect, the present disclosure provides an apparatus for implementing any one of the aforementioned methods.

According to another aspect, the present disclosure provides a computer program product including a computer readable medium having recorded thereon a computer program that when executed on a processor of a computer implements any one of the methods described above.

Other aspects of the present disclosure are also provided.

### BRIEF DESCRIPTION OF THE DRAWINGS

One or more embodiments of the present disclosure will now be described by way of specific example(s) with reference to the accompanying drawings, in which:

FIG. 1 is a schematic block diagram representation of an embodiment of a brain stimulation system in accordance with the present disclosure;

FIG. 2 shows four embodiments of electroencephalogram (EEG) headsets;

FIG. 3 is a schematic block diagram representation of the pre-processing module of FIG. 1;

FIG. 4 is a schematic block diagram representation of functional modules of the brain state inference module of FIG. 1 implemented using Time-Delay Embedded HMM;

FIG. 5 is a schematic block diagram representation of an embodiment of the Stimulation Response Learning Module of FIG. 1;

FIG. 6 is a schematic block diagram representation of a system that includes a general purpose computer on which one or more embodiments of the present disclosure may be practised;

FIG. 7 is a schematic block diagram representation of a system that includes a general smartphone on which one or more embodiments of the present disclosure may be practised;

FIG. 8a illustrates the Extended International 10-20 system for EEG electrode placement, also known as the 10-10 system, showing modified combinatorial nomenclature;

FIG. 8b shows an alternative map of EEG locations, also known as the 10-5 system, with further electrode positions identified in the spaces between the EEG electrode locations of the Extended International 10-20 system;

FIG. 9 is a schematic block diagram representation of a brain stimulation system in accordance with an embodiment of the present disclosure;

FIG. 10 is an illustration of sample EEG signal data captured over 3 seconds; and

FIGS. 11A-C illustrate the relationship from a brain network to a brain network pattern and then to a brain state timecourse.

Method steps or features in the accompanying drawings that have the same reference numerals are to be considered to have the same function(s) or operation(s), unless the contrary intention is expressed or implied.

### DETAILED DESCRIPTION

The present disclosure provides a computer-implemented system and associated method for stimulation of the brain of a mammal. In some embodiments, the system and method are utilised for diagnosing a neurological disorder or psychiatric condition of a human patient. In some embodiments, the system and method are utilised for providing a prognostic indication of the likelihood of an individual human patient with a neurological or psychiatric disorder responding well to a particular treatment. In some embodiments, the system and method are utilised for treating a neurological disorder or psychiatric condition of a human patient.

The brain stimulation system disclosed herein is a closed-loop, non-invasive brain stimulation system. The system: (1) records neurophysiological data of a patient; (2) applies pre-processing to the data; (3) extracts key neural features from this data to determine a current “brain-state” of the patient; (4) utilises artificial intelligence (AI) to determine a stimulation protocol that best elicits or inhibits a particular brain state; and (5) applies brain stimulation to the patient based on the learnt stimulation protocol.

Whilst embodiments are described herein with reference to human patients, the system and method of the present disclosure are applicable to the diagnosis and treatment of mammals broadly, including, but not limited to, dogs, cats, horses, pigs, and primates.

As described above, therapeutic outcome targets refer herein to desired changes in brain states that are sought to be elicited by brain stimulation therapy to achieve a therapeutic goal for a particular patient cohort. However, it is to be noted that such therapeutic outcome targets may relate not only to therapeutic applications, but may equally include clinical applications, such as diagnostic applications and prognostic applications, and monitoring.

Many psychiatric and neurological disorders are linked to abnormalities in brain network activation. Brain networks are distributed regions of cortex that tend to coactivate in unison. A number of canonical brain networks have been characterised that are functionally responsible for higher order cognitive functions, for example the brain's default mode network that coordinates internally oriented modes of cognition such as rumination, mind wandering, episodic memory, and self-referential thought. Abnormal patterns of connectivity in nodes of the default mode network have been implicated in a number of psychiatric illnesses, for example with increased connectivity with subgenual anterior cingulate cortex linked robustly to major depression. This has led to the default mode brain network being characterised as a therapeutic target for a number of psychiatric illnesses, such as depression.

Whereas brain networks have been widely studied using functional magnetic resonance imaging, the expression of brain networks in neurophysiological signals is not widely acknowledged or widely utilised in clinical practice. This is important because neurophysiology, in particular electroencephalography (EEG) reflects a more economically accessible and practical tool for simultaneous integration with emerging forms of therapeutic brain stimulation than traditional methods used for imaging whole-brain networks, such as functional magnetic resonance imaging and positron emission tomography.

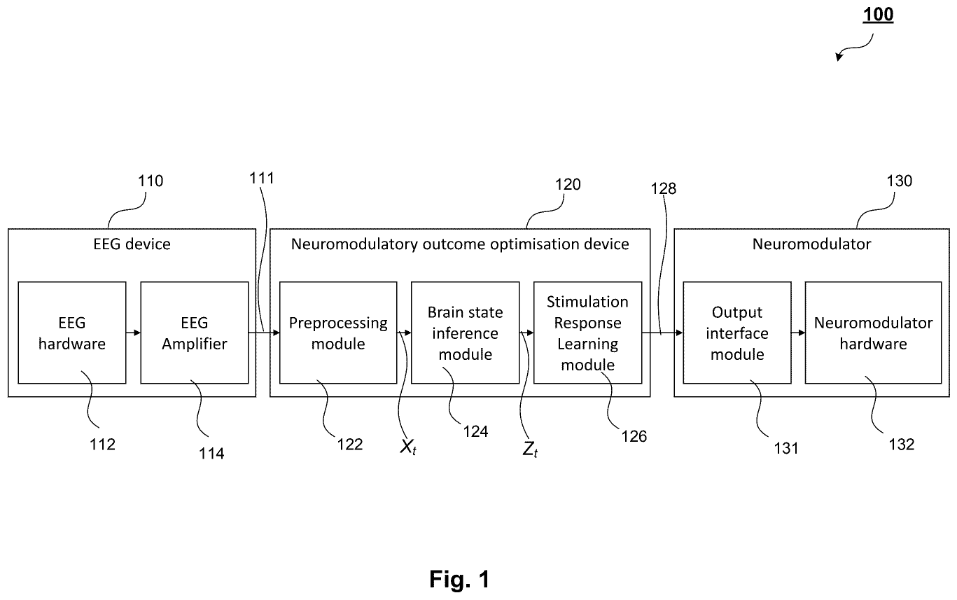

FIG. 1 is a schematic block diagram representation of an embodiment of a brain stimulation system 100 in accordance with the present disclosure. The system 100 includes EEG hardware 110 coupled to a computer-implemented pattern learning device, in the form of a neuromodulatory outcome optimisation device 120, which in turn is coupled to a neuromodulator 130. The EEG hardware 110 may also be referred to as an EEG device 110.

In use, a first step 112 records EEG data from the scalp of a human patient using the EEG hardware 110. The EEG hardware 110 includes one or more EEG sensors 112, which are also known as EEG electrodes, placed on the scalp of the patient, and an EEG amplifier 114 to which the sensors are connected. EEG sensors 112 detect electrical activity from the brain of a subject patient. Placing a plurality of EEG sensors 112 at different locations on the scalp of the patient enables the electrical activity of different parts of the brain to be monitored. In particular, each EEG sensor 112 detects the electrical potential difference between the location of the scalp on which the respective EEG sensor is placed and a reference electrode.

The EEG amplifier 114 then amplifies the detected analog voltage signals from these EEG sensors 112 and digitally samples the detected voltage signals to obtain a digital signal suitable for further transmission and computer processing.

Electrical signals display spatial and spectral patterns that reflect different states of underlying brain activity. Electrical activity in the frequency range up to 4 Hz are referred to as delta waves, and their onset over frontal sensors is understood to support states of heightened internal concentration. Electrical signals in the 4-7 Hz range are referred to as theta waves, and their onset over frontal midline areas is understood to support states of increased cognitive control. Electrical waves in the frequency range of 7-13 Hz are referred to as alpha waves, and their onset over parietal areas supports top-down inhibition of sensory input. Electrical signals in the 14-30 Hz range are referred to as beta waves and their onset over motor areas supports maintenance of the current sensorimotor state. Thus, different frequencies of electrical signals and the application thereof to different locations of a patient are associated with stimulation or support of different neurological states. Other frequency ranges and locations in relation to other neurological states may equally be practised.

In some implementations, the EEG sensors are distributed on a headset that is placed on the head of the patient, such that the EEG sensors are placed in known locations of the scalp of the patient when the visor or headset is worn by the patient. Such headsets may take many different forms, including skull caps, visors, and the like. In other implementations, the EEG sensors are placed as discrete sensors on the head of the patient, with suction caps or the like used to removably attach the sensors to the scalp.

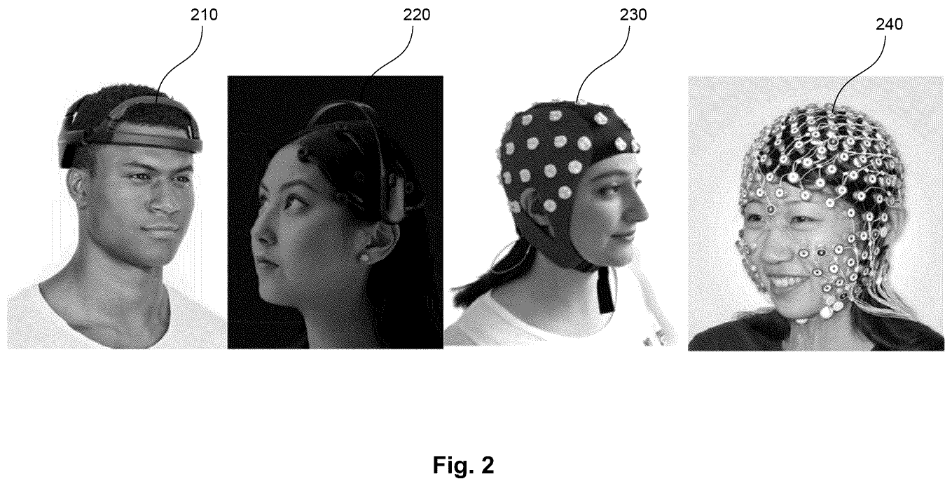

FIG. 2 shows four different embodiments of headsets incorporating EEG sensors that may be utilised in a system of the present disclosure. A first embodiment shows a first headset 210, being the Diadem headset made by Bitbrain (www.bitbrain.com), in which a plurality of EEG sensors are built-in, wherein the headset includes a rigid circular member to surround the skull of the patient when worn. A second embodiment shows a second headset 220, being the EPOC^{X} headset made by Emotiv (www.emotiv.com/epocx), that has a plurality of EEG sensors connected to a retaining device via deformable members. The deformable members allow the individual EEG sensors to be placed so as to make contact with the scalp of the patient.

A third embodiment shows a third headset 230 in the form of a skull cap in which a plurality of EEG sensors are distributed over the skull cap. The third headset 230 is the Waveguard™ EEG cap made by ANT Neuro GmbH (www.ant-neuro.com/products/waveguard_caps). The third headset 230 is retained on the head of the patient using a detachably removable mechanism, such as a strap with a buckle or hook and loop fastener.

A fourth embodiment shows a fourth headset 240 having a plurality of EEG sensors interconnected using a deformable mesh or scaffold, such as may be implemented using silicon rubber or the like. The fourth headset 240 is a high density EEG headset made by Philips (www.usa.philips.com/healthcare/resource-catalog/landing/high-density-eeg). The arrangement of the fourth headset 240 enables a large number of EEG sensors to be placed over a substantial portion of the head of the subject patient.

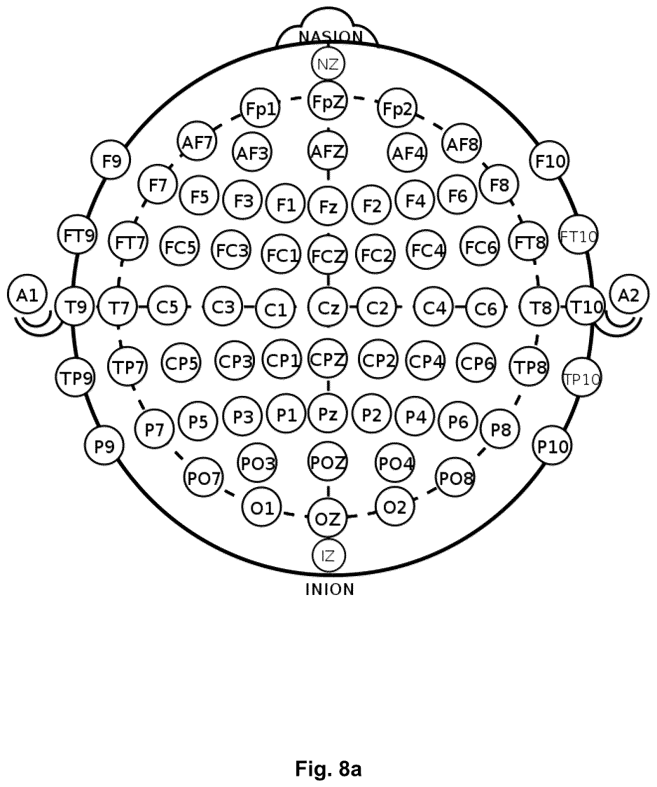

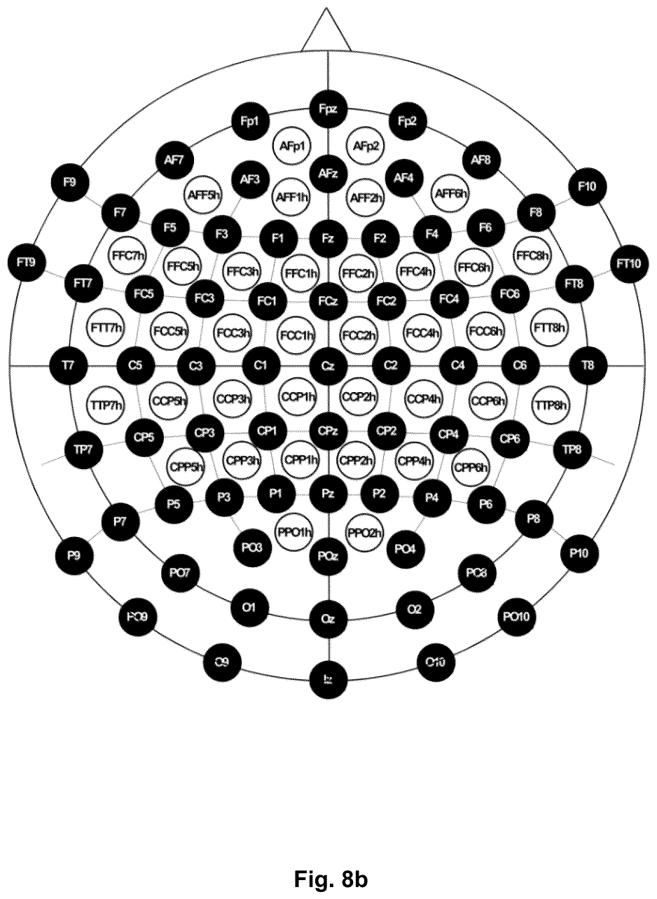

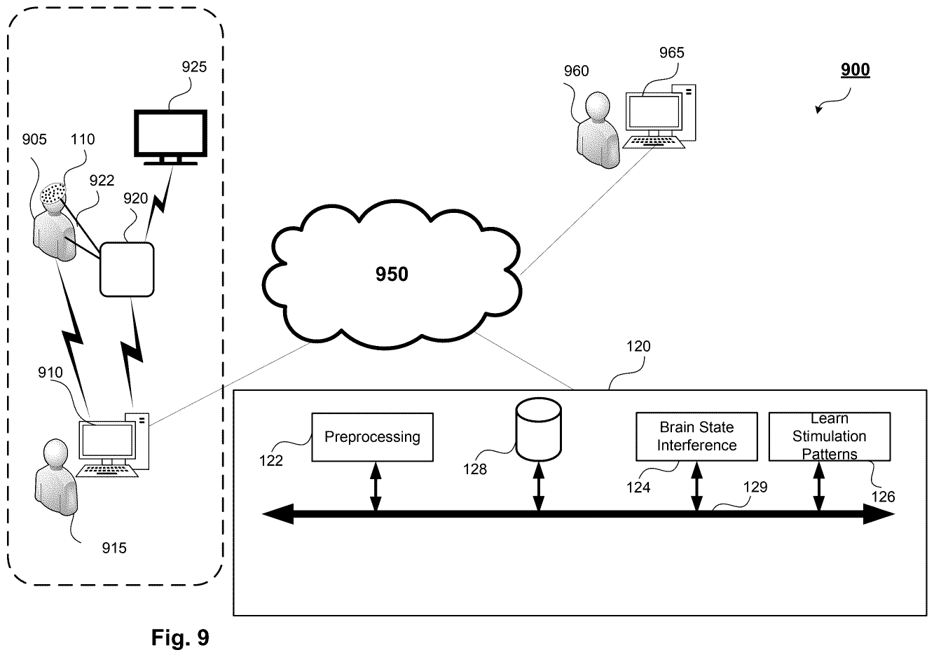

FIG. 8a illustrates the International 10-20 system for EEG electrode placement, showing modified combinatorial nomenclature. FIG. 8b shows an alternative map of EEG locations, with further electrode positions identified in the spaces between the EEG electrode locations of the International 10-20 system.

In each of the embodiments 210, 220, 230, 240, the headset is configured such that the respective EEG sensors are located in predetermined locations on the scalp of the patient when worn. It will be appreciated that other EEG sensors may equally be practised without departing from the spirit and scope of the present disclosure. The montage, or arrangement, of EEG sensors may depend on the stimulation protocol to be applied, the condition to be treated, or the physiology of the patient.

Returning to FIG. 1, the electrical signals 111 detected by the EEG sensors 112 are amplified and digitised before being transmitted to the computing device. This is performed by the EEG amplifier 114. In some implementations, the digitised electrical signals from the amplifier are transmitted to the neuromodulatory outcome optimisation device 120 by a wired communication protocol, such as the Lab Streaming Layer implementation. In other implementations, the digitised electrical signals are transmitted from the EEG device 110 to the neuromodulatory outcome optimisation device 120 via a wireless connection. Any suitable wireless communication protocol may be utilised, including, for example, but not limited to, Bluetooth, Long Range (LoRa), Wi-Fi, Zigbee, Z-Wave, 6LoWPAN, RFID, NFC, 4G/5G, NB-IOT, LTE, and the like.

In some embodiments, the neurophysiological data referred to herein as EEG data may be augmented or replaced by recordings from sensors of another modality. Such modalities include magnetoencephalography (MEG), electromyography (EMG), optically-pumped magnetometer based MEG (OP-MEG), as well as functional near-infrared spectroscopy (fNIRS).

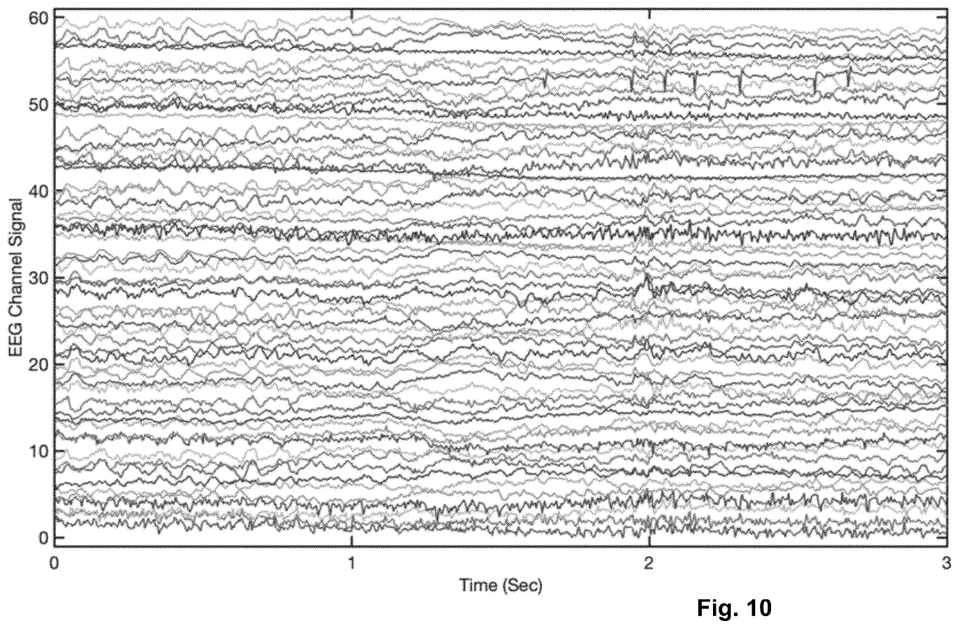

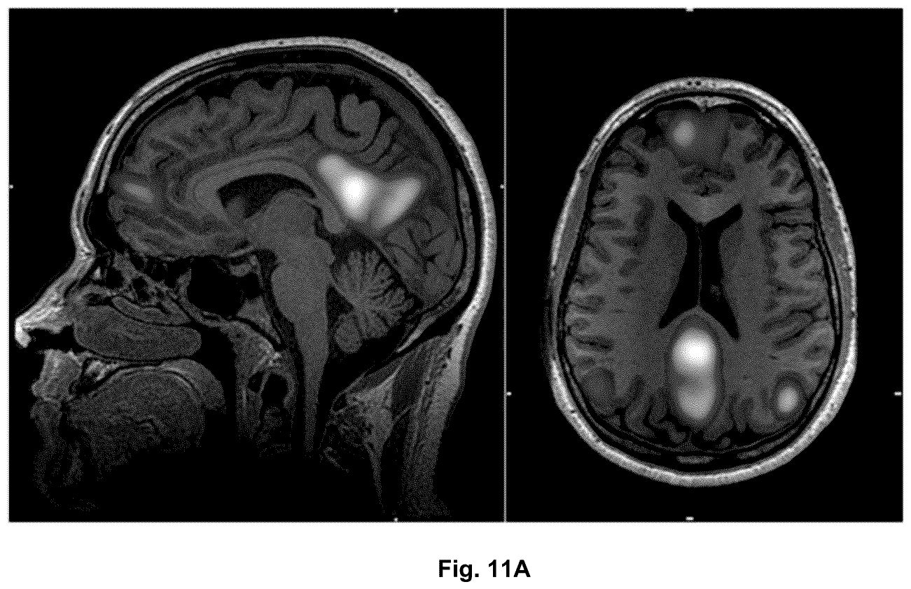

The digital signals output by the EEG hardware 110 are transmitted as EEG signal data 111 to the neuromodulatory outcome optimisation device 120 for storage and processing. FIG. 10 is an example of sample EEG signal data captured over 3 seconds for EEG hardware 110 having 60 EEG sensors 112 that generate signal data for 60 channels. While the neuromodulatory outcome optimisation device 120 is shown as a single functional block, in practice the neuromodulatory outcome optimisation device 120 may be implemented utilising one or more physical computing devices, one or more cloud computing systems, or a combination thereof.

In some implementations, the electrical signals 111 detected by the EEG sensors are amplified and/or digitised before being transmitted to the computing device. In other implementations, the electrical signals 111 are transmitted to the neuromodulatory outcome optimisation device 120 and the neuromodulatory outcome optimisation device amplifies and/or digitises the electrical signals, as required.

A pre-processing module 122 of the neuromodulatory outcome optimisation device 120 pre-processes the detected electrical signals 111. The pre-processing may be utilised to amplify the electrical signals, digitise the electrical signals, buffer the received signals, remove artefacts, format the signals into a predefined format suitable for processing, filter extraneous data, or any combination thereof. A brain state inference module 124 processes the electrical signal data received from the pre-processing module 122 to determine the inferred brain state of the patient.

In some embodiments, the neuromodulatory outcome optimisation device 120 stores a set of predefined brain states, wherein each brain state is associated with a set of brain state attributes. The brain state attributes may include, but are not limited to, brain network patterns and/or brain network dynamics.

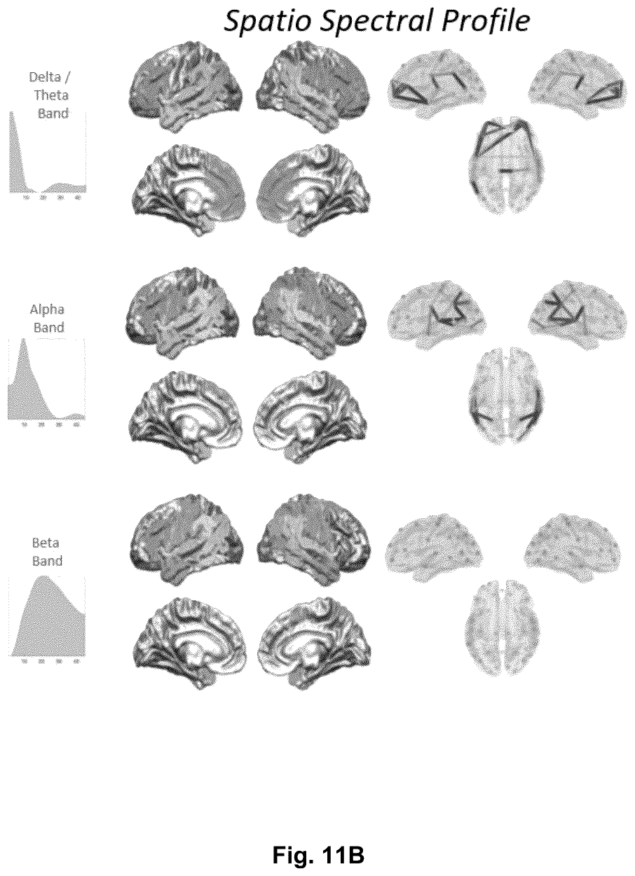

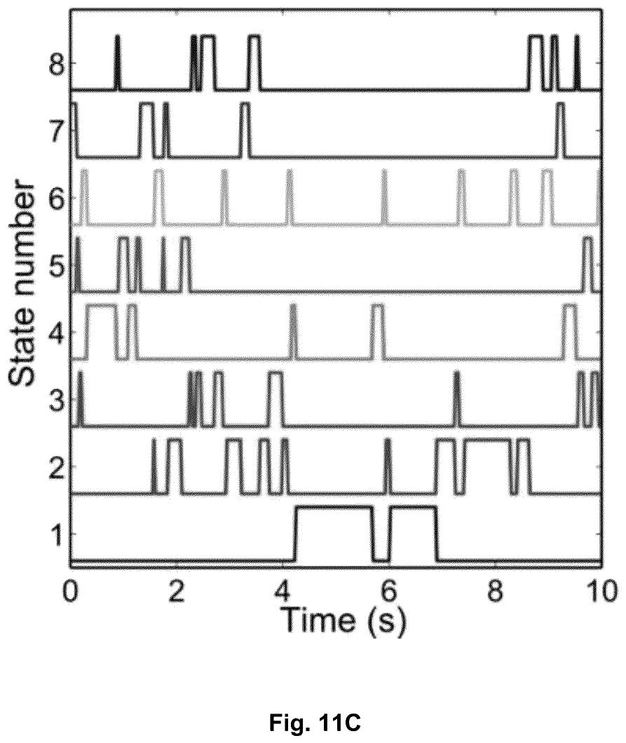

A brain network pattern is a multivariate spatio-spectral distribution that has been identified as the neurophysiological correlate of activation of a particular brain network. The relationship between these is illustrated in FIGS. 11A-C. In this example, activation of the default mode brain network of a patient (as shown in FIG. 11A) has been identified as correlating with a spatio-spectral profile of coherent alpha band oscillations over parietal and lateral-parietal areas at the same time as elevated coherent oscillations between frontotemporal areas in the delta and theta frequency bands (as shown in FIG. 11B). Therefore, the brain network pattern for the default mode network is specified by a mean vector and covariance matrix that mathematically define this pattern over a block of time-embedded and dimensionality reduced neurophysiological data. This information allows us to predict the timing of activation of different brain states, which are understood to reflect an estimate of the time of activation of the true brain network (as shown in FIG. 11C).

Brain state attributes also include brain network dynamics, which reflect the tendency of certain brain networks to activate following the activation of another brain network. For example, the default mode network of the brain has a tendency to activate immediately following activation of the sensorimotor beta network of the brain, whereas the default mode network very rarely activates immediately after activation of the dorsal attention network of the brain. In some embodiments, brain network dynamics are reflected by a Hidden Markov Model with a latent transition probability matrix. This transition probability matrix reflects an elevated probability for the state corresponding to the brain's default mode network to activate immediately following the state corresponding to the brain's sensorimotor beta network, and a reduced probability to activate immediately following the state corresponding to the brain's dorsal attention network.

The neuromodulatory outcome optimisation device 120 determines a current brain state of the patient based on how well the electrical signal data matches the respective brain state attributes associated with the respective brain states.

In some embodiments, the set of brain state attributes associated with each brain state, which may include the associated brain network patterns and/or brain network dynamics, are initially predefined and may be user-defined, based on normative data, or a combination thereof. For example, the set of brain state attributes described above that correspond to activation of the brain's Default Mode Network have been the subject of numerous publications and are well characterised in publicly available datasets, which could be used to set these attributes. Depending on the implementation, brain state attributes associated with any brain state may be updated and modified over time for an individual patient, based on data acquired from that patient.

In some embodiments, the set of brain state attributes associated with each brain state are identified by application of one or more methods described in Australian Provisional Patent Application No. 2023904187.

A Stimulation Response Learning Module 126 utilises artificial intelligence to learn stimulation protocols that best elicit therapeutic outcome targets, such as by exciting or inhibiting one or more brain states, based on the received EEG data 111 and the inferred brain state determined by the brain state inference module 124. In some embodiments, the artificial intelligence is implemented as computer code executing on one or more processors to implement a machine learning algorithm.

In some embodiments, an initial set of training data is utilised to create artificial intelligence models for use in the system. Those models are then updated during use, learning from data as it is acquired from a patient subjected to a stimulation pattern.

The neuromodulatory outcome optimisation device 120 outputs the learnt stimulation patterns 128 to the neuromodulator 130. The neuromodulator 130 includes an output interface module 131 that converts the received learnt stimulation patterns 128 into a form communicable to the neuromodulator 130. The output of the output interface module 131 is presented to a neuromodulatory hardware module 132 that utilises a neuromodulatory interface to apply one or more of the learnt stimulation patterns to elicit or inhibit a brain state in a patient (i.e., the therapeutic outcome target). The neuromodulator 130 triggers changes in brain state dynamics of the patient by providing stimuli to the patient.

In some embodiments, the neuromodulator 130 is implemented using a sound and/or light source that stimulates the brain of a patient via the auditory and visual pathways. The sound and/or light source may be referred to as a neuromodulatory interface and may be implemented, for example, using a display screen, an audio speaker, or a combination thereof. Other forms of intermodulatory interfaces may equally be practised, including, for example, but not limited to, magnetic coils and electrodes. Further, an intermodulatory interface may include a combination of any one or more of the above-mentioned interfaces.

A selected combination of sound and light sources may be utilised to stimulate only auditory pathways, only visual pathways, or a combination of auditory and visual pathways. Such mechanisms of neuromodulation are known as Closed Loop Auditory Stimulation (CLAS) or Audio-Visual Stimulation (AVS). The sound and light stimuli generated by the sound and light sources may vary in duration, amplitude, frequency, and patterns to elicit selected responses from the patient. For example, light stimuli may be presented as pulses of different intensity, frequency, and in different patterns; or alternatively may be presented in pulses at a specific pre-defined frequencies, such as the Gamma (>30 Hz) frequency used in Gamma Entrainment Using Sensory Stimulation (GENUS).

Depending on the intended application, different neuromodulators can be utilised, including Transcranial Magnetic Stimulation (TMS), Transcranial Direct Current Stimulation (TDCS), Transcranial Alternating Current Stimulation (TACS), and Transcranial Ultrasound Stimulation (TUS). Indirect neuromodulators that achieve their modulatory effect on the brain via sensory pathways, such as Peripheral Nerve Stimulation (PNS) or Non-invasive Electrical Pulse Generators (NEPS), may equally be utilised. Such indirect neuromodulators apply electrical pulses to any part of the body, such as the hand or foot of a patient.

It is to be understood that neuromodulatory hardware 132 includes all such hardware components required to implement the neuromodulator. For example, in embodiments in which the neuromodulatory outcome optimisation device 120 controls the timing of stimulation delivered by a fixed TMS neuromodulatory device, the neuromodulatory hardware 132 may include a TMS coil, such as a cooled figure-of-eight coil, a signal generation unit, a cooling unit, an extra power supply, and a fixed TMS positioning arm.

In other embodiments in which the neuromodulatory outcome optimisation device 120 controls the spatial position of robotically-guided TMS neuromodulation, the neuromodulatory hardware 132 may include a TMS coil, such as a cooled figure-of-eight coil, a signal generation unit, a cooling unit, an extra power supply, a robotic TMS positioning arm, a robotic control unit, an optical (e.g. infrared) position sensor or camera, pointer tools and multiple coil trackers.

In such embodiments, it is furthermore understood that the neuromodulatory hardware 132 may also encompass both hardware and software components necessary to administer the neuromodulatory apparatus, such as software that implements robotic control. For example, in some embodiments the output interface module 131 emits a signal reflecting aa desired spatial location, or a desired direction of robotic movement in a two or three dimensional coco-ordinate system. In such embodiments, the neuromodulatory hardware 132 includes a computer processor and neuronavigation software that converts this signal into an appropriate robotic control signal, taking into account patient location, movement and safety, and transmits that signal to the robotic hardware.

The system 100 enables a user to diagnose and treat a range of brain health illnesses, including mental health illnesses (such as depression or anxiety), neurodegenerative illnesses (such as dementia), and neuropathies (such as chronic pain or migraine relief).

Some embodiments are utilised in the diagnosis and treatment of depression. Depression is a mental health illness that is associated with specific brain network patterns. In some such embodiments, the neuromodulator 130 is implemented as a TMS device. TMS is a non-invasive brain stimulation treatment that applies magnetic pulses to a brain of a patient by passing electric current through a magnetic coil placed in relative proximity to the head of the patient. Applying different electric currents to the coil enables a range of different stimuli to be applied to the patient.

In some embodiments, the EEG hardware includes a headset that can be worn by a patient, wherein the headset includes at least two EEG sensors. The EEG sensors (electrodes) may be wet or dry electrodes, or a combination thereof. The headset is configured such that the EEG sensors can be positioned and retained on the scalp of a patient without external support. As described above, FIG. 2 illustrates four embodiments of headsets that may be practised in conjunction with the system of the present disclosure.

One embodiment utilises a high density electrode cap, such as the cap 240 of FIG. 2 being the Geodesic EEG System 400 Research high density EEG headset made by Philips, that uses 256 channels. Such an arrangement of electrodes provides a high standard of data acquisition. Different applications may not require electrodes positioned as extensively across the scalp of the patient, thus allowing less complex headsets to be utilised with fewer sensors and lower power requirements. Depending on the application, different electrode positions may be utilised to acquire signals from particular areas of the brain.

The headset 110 is coupled to the neuromodulatory outcome optimisation device 120 via a wired transmission link, a wireless transmission link, or a combination thereof. In some embodiments, the headset 110 is coupled directly to the neuromodulatory outcome optimisation device 120 via a wired connection, such as a Universal Serial Bus (USB) cable or the like.

In some embodiments, the headset 110 includes a wireless transmitter for transmitting data wirelessly to a compatible wireless receiver in the neuromodulatory outcome optimisation device 120. Such wireless transmitters may utilise, for example, a wireless transmission protocol selected from the group that includes: radiofrequency (RF), Bluetooth, Wi-Fi, Zigbee, Z Wave, 6LowPAN, GRPS/3G/4G/5G/LTE, Near Field Communication (NFC), or the like. Depending on the implementation, the wireless transmitter may be integral with the headset or external to the headset.

The headset 110 includes one or more power sources to power the EEG sensors. Such power sources may include batteries, mains power, or a combination thereof. In some embodiments, the power source is at least one rechargeable battery of sufficient capacity to power the EEG sensors to record data continuously for a clinical session. For example, the battery capacity or power supply may be specified to provide power to the headset for 110 for at least 30 minutes or 1 hour or other defined period. Suitable batteries may include AAA battery cells, AA battery cells, button cell batteries (i.e., CR cells), or the like.

When powered by mains power, the headset 110 is capable of recording data continuously. Depending on the application, different power sources of different capacities may be utilised. In other applications, the headset 110 is powered by an external device, such as the neuromodulatory outcome optimisation device 120 or a laptop computer, via a USB cable or other suitable connection, whereby the headset 110 is able to record data continuously for as long as the headset 110 receives power.

As noted above, the neuromodulatory outcome optimisation device 120 may be implemented using one or more physical computing devices, one or more cloud computing structures, or a combination thereof. The neuromodulatory outcome optimisation device 120 is configured to provide output in the form of stimulation instructions 128 to the neuromodulator 130. In some embodiments, the latency between the incoming EEG signal and the output signal provided to the neuromodulator is 500 ms or less and preferably in the range of 100 ms or less.

One embodiment utilises a general purpose computing device, such as a personal computer or laptop computer, programmed to perform the functions of one or more of the pre-processing module 122, the brain state inference module 124, and the stimulation response learning module 126 so as to realise an improved computing device.

An alternative embodiment implements the neuromodulatory outcome optimisation device 120 or part thereof as a software application (“app”) executing on a mobile phone. In some embodiments, the app communicates via a communications network with a computer server or cloud based computing system. In some embodiments, the software application (“app”) encompasses both the neuromodulatory outcome optimisation device 120 and the neuromodulator 120, with the app user interface also acting as neuromodulatory hardware 132, in particular where the desired neuromodulatory mechanism is CLAS, AVS or GENUS.

FIG. 9 is a schematic block diagram representation of a brain stimulation system 900 in accordance with an embodiment of the present disclosure. The system 900 includes a computer-implemented neuromodulatory outcome optimisation device 120, corresponding to the device 120 of FIG. 1.

The neuromodulatory outcome optimisation device 120 is coupled to a communications network 950. The communications network 950 may comprise one or more wired communications links, wireless communications links, or any combination thereof. In particular, the communications network 950 may include a local area network (LAN), a wide area network (WAN), a telecommunications network, or any combination thereof. A telecommunications network may include, but is not limited to, a telephony network, such as a Public Switch Telephony Network (PSTN) or a cellular mobile telephony network, the Internet, or any combination thereof.

Each of the pre-processing module 122, the brain state interference module 124 and learning module 126 communicate via a bus 129. The neuromodulatory outcome optimisation device 120 also includes a computer readable storage medium 128 for storing a set of brain states, brain state attributes including brain network patterns and brain network dynamics, patient data, and machine learning training data and models.

The system 900 also includes a patient 905 wearing an EEG headset 110 that is coupled to a controlling computing device 910 operated by a user 915. In some embodiments, the patient 905 and the user 915 are the same person, such as in a self-administered system implemented in a home setting. Depending on the implementation, the EEG headset 100 is coupled to the controlling computing device 910 via one or more wired or wireless communications links, including Bluetooth, Wi-Fi, Ethernet, and the like.

The controlling computing device 910 sends signals to a neuromodulatory device 920 to apply stimuli to the patient 905. The stimuli are applied via a neuromodulatory interface. In some embodiments, such as shown in FIG. 9, the neuromodulatory interface is implemented using one or more electrodes or magnetic coils 922 placed on or adjacent to the patient 905. Depending on the application and implementation, the stimulating electrodes or magnetic coils 922 may be placed at one or more of the spine, brain, peripheral nerves, or any combination thereof. Where the neuromodulatory interface is implemented using electrodes, the neuromodulatory device 920 utilises a pulse generator and power source to apply stimuli via the stimulating electrodes.

In other embodiments, the neuromodulatory interface is implemented using a display screen, an audio speaker, magnetic coils, or any combination thereof. In such embodiments, the neuromodulatory device 920 controls the neuromodulatory interface to apply the relevant stimuli. For example, FIG. 9 also shows a display screen 925 that is coupled to the neuromodulatory device 920, wherein the neuromodulatory device 920 controls output on the display 925 to deliver visual stimuli to the patient 905. Where the display 925 is equipped with an audio speaker, the neuromodulatory device 920 controls the output on the display 925 and the audio speaker to deliver visual stimuli, audio stimuli, or a combination thereof to the patient 905.

In arrangements in which the neuromodulatory device 920 is coupled to more than one neuromodulatory interface 922, 925, the neuromodulatory device 920 controls the neuromodulatory interfaces 922, 925 to deliver any combination of available stimuli to the patient 905.

Depending on the implementation, the neuromodulatory device 920 may be coupled to the controlling computing device 920 via one or more wired and/or wireless communications links. In some embodiments, the controlling computing device 910 and the neuromodulatory device 920 are integrated into a single device. In some embodiments, the display device 925 and the controlling computing device 910 are integrated into a single device,

The EEG headset 110 detects electrical potential detected from the patient 905 in response to stimuli and transmits electrical signals to the control computing device 910. In the example of FIG. 9, the control computing device 910 transmits the electrical signals via the communications network 950 to the neuromodulatory outcome optimisation device 120. The neuromodulatory outcome optimisation device 120 processes the signals and then sends control commands to the control computing device 910, wherein the control commands correspond to stimulus patterns to be applied to the patient 905 by the EEG headset.

In some embodiments, the control computing device 910 and neuromodulatory outcome optimisation device 120 are co-located with each other or even integral with each other.

The system 900 also includes a second computing device 965 coupled to the communications network 950 and accessed by a second user 960. The second user 960 utilises the second computing device 965 to communicate with the neuromodulatory outcome optimisation device 120 to view patient data, update training data and models, and the like. In some implementations, the neuromodulatory outcome optimisation device 120 has an associated web interface in the form of a dashboard to enable a user to view and access data and controls pertaining to the neuromodulatory outcome optimisation device 120.

In some implementations, any one or more of the control computing device 910, the second computing device 965, and the neuromodulatory outcome optimisation device 120 are implemented using one or more of a personal computer, laptop computer, tablet computing device, mobile phone, or the like.

Returning to FIG. 1, the pre-processing module 122 pre-processes the received EEG data 111 to ensure that the data is as informative as possible of the detected underlying brain state of the patient. In particular, the detected EEG signal data 111 contains both neural components and non-neural components. The non-neural components are derived from non-neural sources, such as muscle artefacts and heartbeats. The pre-processing removes the non-neural components of the received EEG signal data 111 such that the residual signal is as reflective as possible of the underlying brain activity.

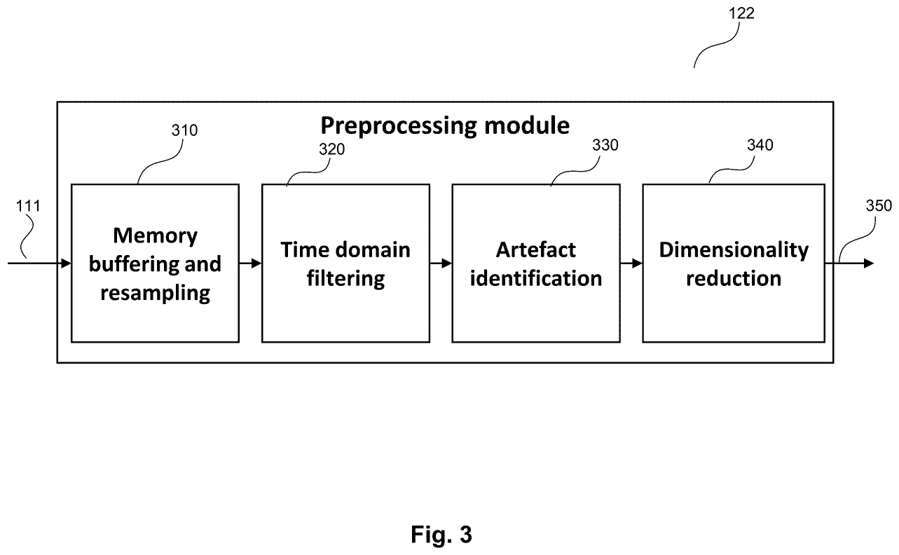

FIG. 3 is a schematic block diagram representation of one embodiment of the pre-processing module 122 of FIG. 1. The pre-processing module 122 receives the EEG signal data 111 from the EEG hardware 110. The EEG signal data 111 is processed by a memory buffer 310, which serves as a temporary storage mechanism to ensure a steady and consistent flow of transmitted samples by accommodating variations in data arrival rates. In some implementations, this memory buffer module also performs resampling with polyphase anti-aliasing filtering such that the samples are transmitted out of the memory buffer at a lower sampling rate to that which they were received. In some embodiments, the sampling rate after downsampling is 100 samples per second. The actual sampling rate will depend on the application and may be in the range of 50 samples per second to 16,000 samples per second, depending on needs and processing capabilities.

In some embodiments, the EEG signal data is then filtered by a time domain filter 320. The time domain filter 320 applies linear time invariant filtering to the data recorded from each channel/electrode, in order to remove high frequency noise and low frequency line drift. Some embodiments utilise a 3^{rd} order Butterworth filter with a passband of 1-45 Hz.

Some embodiments pass the signal to an optional artefact identification module 330, which applies an algorithm to the filtered signal to identify artefacts, irrespective of whether that signal has been filtered. One embodiment computes the standard deviation of the signal across all channels at each point in time and classifies the signal as an artefact wherever this standard deviation exceeds the 99th percentile observed from configuration data. The artefact identification module 330 then removes identified artefacts. In some embodiments, the artefact identification module 330 removes part of the signal that is an artefact. In other embodiments, the artefact identification module 330 removes an entire signal or portion of signal, such as by sending an indication to downstream processing not to process the signal until the artefact has passed.

In some embodiments, a dimensionality reduction module 340 processes the remaining signal to reduce the inherent dimensionality of the signal. In one embodiment, the dimensionality reduction module 340 applies principal component analysis to the configuration data to identify the set of linear loadings that capture 90% of the data variance. In one or more embodiments, the dimensionality reduction module applies a suitable source estimation method, such as linearly constrained minimum variance beamforming followed by parcellation and source leakage correction to identify the set of linear loadings that map the EEG data into a common reference space with lower dimensionality. In one or more embodiments, the dimensionality reduction module 340 applies a spatial Laplacian transform to the EEG data. The output of the dimensionality reduction module 340 is presented as output signal Xₜ to be processed by the brain state inference module 124.

Returning to FIG. 1, the brain state inference module 124 processes the signal Xₜ, received from the pre-processing module 122 as a vector of pre-processed data over a set of channels, to determine what brain network is active at a given point in time, based on statistical analysis of the pre-processed data signal Xₜ. In some embodiments, the brain state inference module 124 transforms the received pre-processed vector Xₜ to reflect both spatial and spectral patterns (by time delays, concatenation and linear transformation), then solves a Bayesian inverse problem for inferring the current active brain state (by likelihood estimates, prior computation and softmax functions). The brain state inference module 124 outputs an inferred brain state Zₜ, as a vector, for a given timepoint.

Some embodiments of the brain state inference module 124 utilise Time-Delay Embedded Hidden Markov Model (HMM) to link data to identified brain networks. This implementation is favoured due to established evidence that the states inferred correspond to brain network activation of physiological, behavioural, and clinical relevance. As shown below, this model assumes Markovian state dynamics and a Gaussian distribution over the raw sensor data. Nonetheless, other embodiments may include different assumptions, such as state dynamics modelled using recurrent neural networks; state dynamics modelled using temporal convolutional neural networks; non-Gaussian distributions over the raw data; and the application of nonlinear transformations to the raw data, such as Short-Time Fourier Transforms, wavelet transforms or other methods for estimating bandlimited power and coherence.

It will be appreciated that different embodiments may omit some of the functional modules 310, 320, 330, 340, of FIG. 3. Further, the order of the functional modules 310, 320, 330, 340 may change, depending on the implementation, without departing from the spirit and scope of the present disclosure.

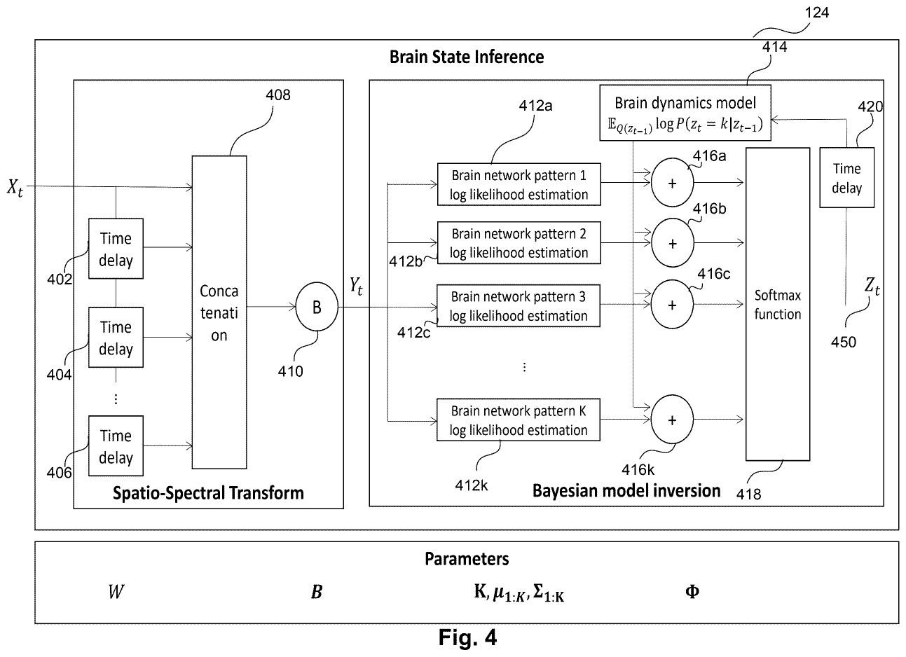

FIG. 4 is a schematic block diagram representation of functional modules of a brain state inference module 124 implemented using Time-Delay Embedded HMM. The brain state inference module 124 implements a set of computations that are derived from an underlying mathematical model of how the recorded data relate to activation of brain networks. The assumptions of the model, which motivate each step in the brain state inference module, include:
When different brain networks activate, the respective brain networks result in scalp potentials that differ in both the spatial and spectral properties of the resulting timeseries.
It is assumed that we have already learned the different spatial and spectral patterns unique to the activation of each brain network.
Standard assumptions of Hidden Markov Models are applied, specifically:
   (1) that these brain networks are mutually exclusive with respect to time; and
   (2) the sequence of brain states forms a Markov chain, i.e. that a state zₜ is conditionally independent of zₜ₋ₙ∀n>1, if zₜ₋₁ is known.

Based on these assumptions, the brain state inference module 124 is implemented to answer a question at each time step. If the brain network that was activated one timestep prior (denoted by zₜ₋₁) is known, and the current spatial and spectral pattern of scalp potentials (denoted by Yₜ) are also known, then the question to be answered is what is the most likely brain network that is currently active (denoted by zₜ).

The embodiment of the brain state inference module 124 depicted in FIG. 4 answers this question in a Bayesian manner:

$$P ( {z}_{t} \vert {z}_{t - 1} , {Y}_{t} ) = \frac{P ( {Y}_{t} \vert {z}_{t} ) P ( {z}_{t} \vert {z}_{t - 1} )}{P ( {Y}_{t} \vert {z}_{t - 1} )} \qquad Eqn ( 1 )$$

Eqn (1) is solved by the embodiment of the brain state inference module 124 shown in FIG. 4 using time delay embedding, dimensionality reduction, and state likelihood computation.

As shown in FIG. 4, the brain state inference module 124 receives the vector signal Xₜ and performs time delay embedding to create a data vector that captures both the spatial patterns expressed over different channels as well as the spectral patterns, such as the frequency of a brainwave, which can only be observed by looking at the relationships between data over successive timepoints.

Given a [P×1] vector Xₜ output from the pre-processing unit 122 at time t, where P is the number of EEG channels, the first step of the Time Delay Embedded HMM is to create a time embedding of the data. This constructs a new vector Xₜ of dimension [PW×1] where W is the length of the embedding.

The entire [P×1] vector Xₜ is passed through each time delay element, such that the output of 402 is a [P×1] vector Xₜ₋₁; the output of 403 is a [P×1] vector Xₜ₋₂, etc.

The concatenation module 408 has W different vector inputs, being specifically Xₜ, Xₜ₋₁, Xₜ₋₂, . . . X_{t-W+1}. The concatenation module 408 then outputs a vector \hat{X}ₜ of dimension [PW×1], where \hat{X}ₜ=[Xₜ; Xₜ₋₁; Xₜ₋₂; . . . X_{t-W+1}] (i.e., the row-wise concatenation of the input vectors).

The new vector \hat{X}ₜ is highly dimensional and is expected to contain much information that is superfluous or redundant. Accordingly, the dimensionality reduction module 410 projects the new vector \hat{X}ₜ to a lower dimensional vector Yₜ of dimension [Q×1]. In some embodiments, this is a linear dimensionality reduction operation, such that Yₜ=B\hat{X}ₜ, where B is a linear operator of dimension [Q×PW] such that Yₜ reflects both the spatial and short term spectral profile of the neurophysiological signal. Referring to FIG. 4, the vector \hat{X}ₜ is operated on by the B operator 410 to produce Yₜ. We refer to the group of operations performed to map the vector Xₜ to the vector Yₜ as the spatio-spectral transform.

In some embodiments, the matrix B is learned through an appropriate method such as principal component analysis applied to some reference data, such that the output Yₜ contains the first W principal components of the data. In other embodiments, the matrix B is learned through alternative methods such as those outlined in Australian Provisional Patent Application No. 2023904187.

In one embodiment, the processing of the spatio-spectral transformed data then assumes a Hidden Markov Model with a multivariate Gaussian observation model:

$$P ( {Y}_{t} \vert {z}_{t} = k ) = N ( {μ}_{k} , {\sum }_{k} ) \qquad Eqn ( 2 )$$

where Zₜ is the value of the latent state at timepoint t, μₖ is the mean and Σₖ is the covariance matrix for state k that is learned by an appropriate method, as described below.

Following the standard approach for real time HMMs, the process of inferring which brain state is active at time t is given by:

$$P ( {z}_{t} = k \vert {Y}_{t} , {z}_{t - 1} ) = \frac{P ( {Y}_{t} \vert {z}_{t} = k ) P ( {z}_{t} = k \vert {z}_{t - 1} )}{P ( {Y}_{t} \vert {z}_{t - 1} )} \qquad Eqn ( 3 )$$

Some embodiments apply the variational Bayesian approximation to evaluate this, such that the approximate state probability at each timestep Q(zₜ=k)≈P(zₜ=k|Yₜ,zₜ₋₁) is given by:

$$log Q ( {z}_{t} = k ) = {𝔼}_{Q ( {z}_{t - 1} )} log \frac{P ( {Y}_{t} \vert {z}_{t} = k ) P ( {z}_{t} = k \vert {z}_{t - 1} )}{P ( {Y}_{t} \vert {z}_{t - 1} )} \qquad Eqn ( 4 )$$

Where [symbol] denotes the expectation with respect to the previous timestep's approximate state probability. Referring to FIG. 4, module 412 computes the value of log P(Yₜ|zₜ=k), and module 414 computes the value [symbol] log P(zₜ=k|zₜ₋₁).

In some embodiments, the module 414 implements a model of temporal dynamics that is non-stationary or informed by patient meta-data, for example computing a value of [symbol] log P(zₜ=k,ψ|zₜ₋₁) for some latent variable CD modelled such as by the method described in Australian Provisional Patent Application No. 2023904187.

The final output of the Brain State Inference Module 124 is a [K×1] vector \hat{Z}ₜ, the kth entry of which reflects the probability that state k is active at time t, and is equal to the softmax function implemented by module 418, such that:

$$Q ( {z}_{t} = k ) = \frac{{e}^{P ( {Y}_{t} \vert {z}_{t} = k ) + {𝔼}_{Q ( {z}_{t - 1} )} logP ( {z}_{t} = k \vert {z}_{t - 1} )}}{{\sum }_{j = 1}^{K} {e}^{P ( {Y}_{t} \vert {z}_{t} = j ) + {𝔼}_{Q ( {z}_{t - 1} )} logP ( {z}_{t} = j \vert {z}_{t - 1} )}} \qquad Eqn ( 5 )$$

In the example of FIG. 4, there are K known brain states stored in the brain state inference module 124. For each of the K brain states, the system stores an associated value for μₖ and Σₖ, as well as a [K×K] state transition probability matrix. These values may be updated over time, in some embodiments through the use of the method described in Australian Provisional Patent Application No. 2023904187.

In some embodiments, module 418 instead computes the hardmax function, such that the final output it has a single entry which has probability 1 and all other entries with probability 0.

In other embodiments, the module 414 implements a model of temporal dynamics that includes a memory component with capacity to store the value of states beyond the immediately preceding timestep, i.e. prior to zₜ₋₁. This relaxes the Markov Assumption and can be implemented for example with long short term memory architectures as outlined in Gohil, Higgins et al 2022; (https://doi.org/10.1016/J.neuroimage.2022.119595).

We refer to the group of operations performed to map the vector Yₜ to the inferred brain state Zₜ as the Bayesian model inversion.

This value of \hat{Z}^{t} is the output of the brain state inference module 124 at each timepoint t passed on to the Stimulation Response Learning Module 126 and corresponds to an inferred brain state probability that has been determined, from a set of K predefined brain states, for each point in time t.

The Stimulation Response Learning Module 126 receives the current brain state and outputs a desired action. In some embodiments, the Stimulation Response Learning Module 126 utilises a reinforcement learning algorithm that optimises the stimulation protocol to apply to a patient based on a limited level of exploration of how that patient responds to applied stimuli. Specifically, this module takes as input an inferred brain state probability \hat{Z}ₜ (i.e., the output of the previous module) and outputs an instruction θ to the downstream neuromodulator.

The output instructions could be as simple as an instruction to turn stimulation on or off, but could also be more nuanced instructions such as a new setting for the stimulation parameters, such as the magnitude, frequency, or duration of stimulation, a repeating pattern of stimuli (such as repeating sounds and/or light pulses for audio-visual stimuli) or any combination thereof. In some embodiments, the reinforcement learner module learns the stimulation protocol through three steps: a reward function step, a learning algorithm step, and a policy implementation step.

In some embodiments, the output instructions are used to change neuromodulatory interface or to combine two or more intermodulatory interfaces. For example, if a first set of stimuli was applied by magnetic coils, the output instructions may change the neuromodulatory interface to an audiovisual stimulus or to stimuli electrodes, or any combination thereof. Alternatively, an initial visual stimulus may be augmented by an audio stimulus, stimuli electrodes, magnetic coils, or any combination or order thereof.

Some embodiments determine the appropriate stimulation protocol by first constraining the action set to a binary variable—specifically, this means limiting the scope of allowable actions to a simple on/off instruction, such as an instruction whether the neuromodulator is on or off. This is denoted by constraining θ∈{0,1}.

In some embodiments, the method then proceeds with a Q-learning algorithm, as outlined in greater detail below. Q-learning is one of the simplest and most reliable reinforcement learning algorithms. Furthermore, Q-learning is well matched to the current best-known brain state inference procedure, as the discrete and mutually exclusive state output, combined with binarised action outputs, provides a relatively low dimensional Markov Decision Process for which Q-learning is particularly well suited. Nonetheless, other embodiments may use alternate reinforcement learning paradigms, such as a state-action-reward-state-action (SARSA) algorithm, temporal difference learning algorithm, actor-critic methods, Monte Carlo methods, and deep reinforcement learning methods.

Crucial to any reinforcement learning implementation is the reward function, that defines which outcomes are rewarded and which outcomes are penalised. This is the basis by which the Stimulation Response Learning Module learns which actions to perform in which state.

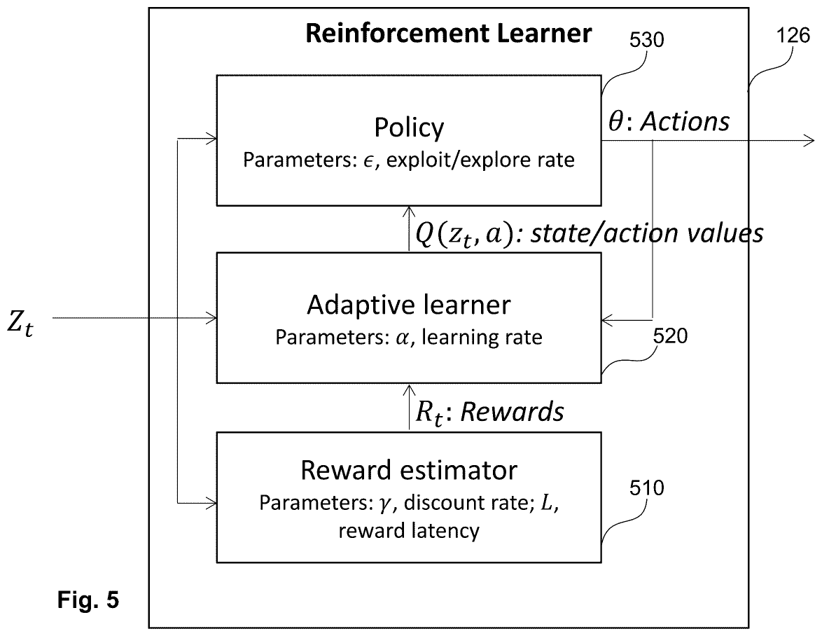

FIG. 5 is a schematic block diagram representation of an embodiment of the Stimulation Response Learning Module 126. The Stimulation Response Learning Module 126 receives as an input an inferred brain state 450 for a timepoint t from the brain state inference module 124 and produces and outputs a protocol instruction θ to the neuromodulator 130. Internally, the machine passes signals between a Reward Estimation Module 510, an Stimulation Response Learning Module 520 and a Policy Implementation Module 530.

In this embodiment, it is assumed that rewards are evaluated by the Reward Estimation Module 510 up to τ timesteps into the future, according to the following formula:

$${R}_{t} = {\sum }_{i = 0}^{τ - L} {γ}^{i} {\hat{Z}}_{t + L + i}^{T} r \qquad Eqn ( 6 )$$

where \hat{Z}ₜ is the output of the brain state inference module at timepoint i, and r is the parameter defining which states are rewarded, of dimension [K×1]. γ is a discount factor taking values 0<γ<1, which downweights actions further into the future, and L is an integer taking values L∈[1,τ] allowing an offset for neuromodulator latency (i.e., so that the reward ignores patterns of brain activity that occur after the action signal is generated but before it would actually have any effect on the brain).

The Stimulation Response Learning Module 520 then learns the relationship between actions and rewards. In some embodiments, this is implemented with Q-learning, a model-free reinforcement learning algorithm that learns the value of an action taken in any particular state. Specifically, Q-learning involves learning a look-up table of values assigned to actions in a particular state, where value corresponds to the expected future rewards that would derive from this particular action (i.e., the expectation of Rₜ given the current state).

In this embodiment, the Stimulation Response Learning Module 520 involves a matrix of parameters Q of dimension Kx A, where A is the number of possible action values (i.e., in the binary case where the reinforcement learner controls whether stimulation is on or off, A=2). Q(i; j) denotes the i,jth entry of this matrix, which corresponds to the expected value of rewards obtained by taking action j during state i:

$$Q ( i ; j ) = 𝔼 [ P ( {R}_{t} \vert {z}_{t} = i , {θ}_{t} = j ) ] \qquad Eqn ( 7 )$$

The values of this matrix are initially populated from a training procedure, which will be described below. The matrix values are then updated by temporal difference learning, according to the following algorithm:

$$Q ( {z}_{t} , {a}_{t} ) ← ( 1 - α ) Q ( {z}_{t} , {a}_{t} ) + α ( {R}_{t} + γ {max}_{a} Q ( {z}_{t + L} , a ) ) \qquad Eqn ( 8 )$$

Finally, this embodiment implements a Policy Implementation Module 530. The Policy Implementation Module receives as input: (1) a current brain state, and (2) the learning algorithm's expected returns associated with different actions in different states, and outputs the selected action θₜ taken at timepoint t 128.

In some embodiments of the system and method described herein, the Policy Implementation Module 530 applies the epsilon-greedy policy, which provides for constant exploration of the state-action space. This behaviour is desirable in our desired application, given the expectation of stimulus habituation, namely that a person's response to the neuromodulatory stimulus may evolve over time.

Algorithm 1: Epsilon Greedy Policy

| input | : Q: a Q table of expected rewards for state-action pairs; zₜ, the current state; ϵ, |
|---|---|
| | parameter controlling exploitation / exploration tradeoff |
| output | : Action instruction θ |

for t < T do

| \| | draw n ϵ [0, 1] from a uniform distribution if n < ϵ then | |
|---|---|---|
| \| | \| | θ ← randomly selected action |
| \| | else | |
| \| | \| | θ ← maxₙ Q(zₜ, α) |

end

In order for the above-described system and method to work, it is necessary to initialise the system by setting values for the parameters in Table 1, such that the brain states correspond to therapeutic outcome targets and the stimulation parameters are effective.

TABLE 1

| Parameter | Description | Module |
|---|---|---|
| a, b | Filter coefficients | Preprocessing |
| P | Dimensionality of Xₜ | Preprocessing |
| W | Time delay length | Brain state inference |
| Q | Dimensionality of Yₜ | Brain state inference |
| B | Spatio spectral transform | Brain state inference |
| | operator | |
| K | Number of states | Brain state inference |
| μ_{1:K} | State mean vectors | Brain state inference |
| Σ_{1:K} | State covariance matrices | Brain state inference |
| Φ | Transition Probability | Brain state inference |
| | parameters | |
| γ | Reward discount rate | Stimulation Response Learning |
| L | Reward latency | Stimulation Response Learning |
| α | Learning rate | Stimulation Response Learning |
| ϵ | Exploit/explore rate | Stimulation Response Learning |

Different training approaches may be practised. Some embodiments entail the use of: (i) Publicly available EEG datasets of clinical populations, (ii) configuration recordings for each new patient, (iii) computations from theoretical models, or any combination thereof.

The output signal 128 of the Reinforcement Learner Module 126 is then communicated to an Output Interface Module 131. The output interface module 131 converts this signal into a form communicable to the neuromodulator (which could include CLAS, AVS, GENUS, TMS, TDCS, TACS, TUS, PNS or NEPS neuromodulation) and transmits that signal to the neuromodulator.

The brain stimulation system of the present disclosure may be practised using a computing device, such as a general purpose computer or computer server that is programmed to perform one or more of the functions shown and described in relation to FIGS. 1, 3-5, thus giving rise to a new and improved computing device.

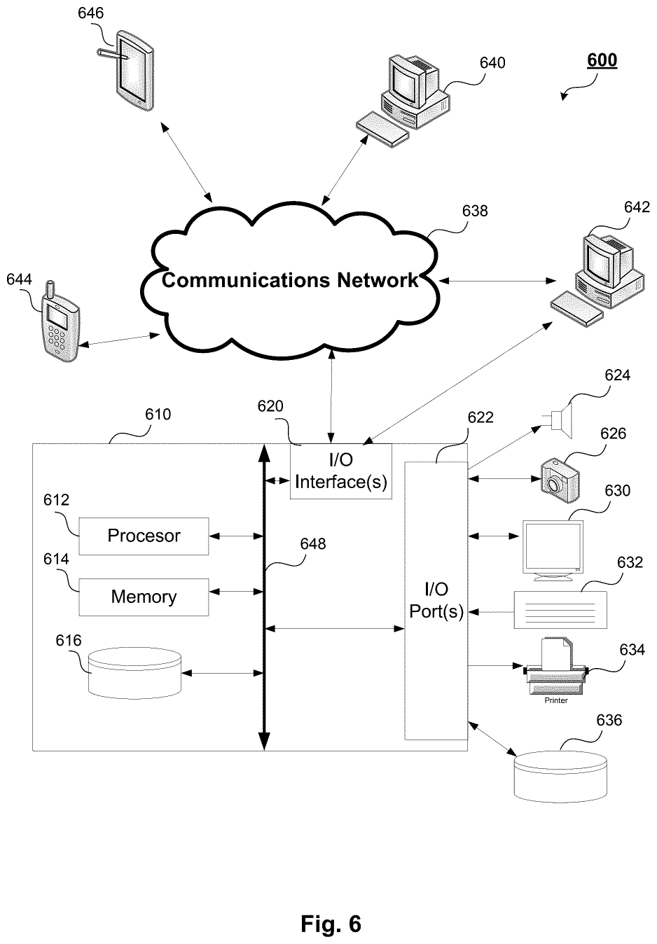

FIG. 6 is a schematic block diagram of a system 600 that includes a general purpose computer 610. The general purpose computer 610 includes a plurality of components, including: a processor 612, a memory 614, a storage medium 616, input/output (I/O) interfaces 620, and input/output (I/O) ports 622. Components of the general purpose computer 610 generally communicate using one or more buses 648.

The memory 614 may be implemented using Random Access Memory (RAM), Read Only Memory (ROM), or a combination thereof. The storage medium 616 may be implemented as one or more of a hard disk drive, a solid state “flash” drive, an optical disk drive, or other storage means. The storage medium 616 may be utilised to store one or more computer programs, including an operating system, software applications, and data. In one mode of operation, instructions from one or more computer programs stored in the storage medium 616 are loaded into the memory 614 via the bus 648. Instructions loaded into the memory 614 are then made available via the bus 648 or other means for execution by the processor 612 to implement a mode of operation in accordance with the executed instructions.

One or more peripheral devices may be coupled to the general purpose computer 610 via the I/O ports 622. In the example of FIG. 6, the general purpose computer 610 is coupled to each of a speaker 624, a camera 626, a display device 630, an input device 632, a printer 634, and an external storage medium 636. The speaker 624 may be implemented using one or more speakers, such as in a stereo or surround sound system. In the example in which the general purpose computer 610 is utilised to implement one or more of the functions of a brain stimulation system, one or more peripheral devices may relate to the EEG hardware 100 of FIG. 1 connected to the I/O ports 622 either wirelessly or by wired connection.

The camera 626 may be a webcam, or other still or video digital camera, and may download and upload information to and from the general purpose computer 610 via the I/O ports 622, dependent upon the particular implementation. For example, images recorded by the camera 626 may be uploaded to the storage medium 616 of the general purpose computer 610. Similarly, images stored on the storage medium 616 may be downloaded to a memory or storage medium of the camera 626. The camera 626 may include a lens system, a sensor unit, and a recording medium.

The display device 630 may be a computer monitor, such as a cathode ray tube screen, plasma screen, or liquid crystal display (LCD) screen. The display 630 may receive information from the computer 610 in a conventional manner, wherein the information is presented on the display device 630 for viewing by a user. The display device 630 may optionally be implemented using a touch screen to enable a user to provide input to the general purpose computer 610. The touch screen may be, for example, a capacitive touch screen, a resistive touchscreen, a surface acoustic wave touchscreen, or the like.

The input device 632 may be a keyboard, a mouse, a stylus, drawing tablet, or any combination thereof, for receiving input from a user. The external storage medium 636 may include an external hard disk drive (HDD), an optical drive, a floppy disk drive, a flash drive, solid state drive (SSD), or any combination thereof and may be implemented as a single instance or multiple instances of any one or more of those devices. For example, the external storage medium 636 may be implemented as an array of hard disk drives.

The I/O interfaces 620 facilitate the exchange of information between the general purpose computing device 610 and other computing devices. The I/O interfaces may be implemented using an internal or external modem, an Ethernet connection, or the like, to enable coupling to a transmission medium. In the example of FIG. 6, the I/O interfaces 622 are coupled to a communications network 638 and directly to a computing device 642. The computing device 642 is shown as a personal computer, but may equally be practised using a smartphone, laptop, or a tablet device. Direct communication between the general purpose computer 610 and the computing device 642 may be implemented using a wireless or wired transmission link.

The communications network 638 may be implemented using one or more wired or wireless transmission links and may include, for example, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a mobile telephone cellular network, a short message service (SMS) network, or any combination thereof. The general purpose computer 610 is able to communicate via the communications network 638 to other computing devices connected to the communications network 638, such as the mobile telephone handset 644, the touchscreen smartphone 646, the personal computer 640, and the computing device 642.

One or more instances of the general purpose computer 610 may be utilised to implement one or more functions of the neuromodulatory outcome optimisation device 120 of FIG. 1 to implement a brain stimulation system in accordance with the present disclosure. In such an embodiment, the memory 614 and storage 616 are utilised to store data relating to brain states, parameters from Table 1, algorithms corresponding to Equations 1 to 7, user interface templates, and the like. Software for implementing the brain stimulation system is stored in one or both of the memory 614 and storage 616 for execution on the processor 612. The software includes computer program code for implementing method steps in accordance with the functional modules described herein, particularly with reference to FIGS. 1 to 5.

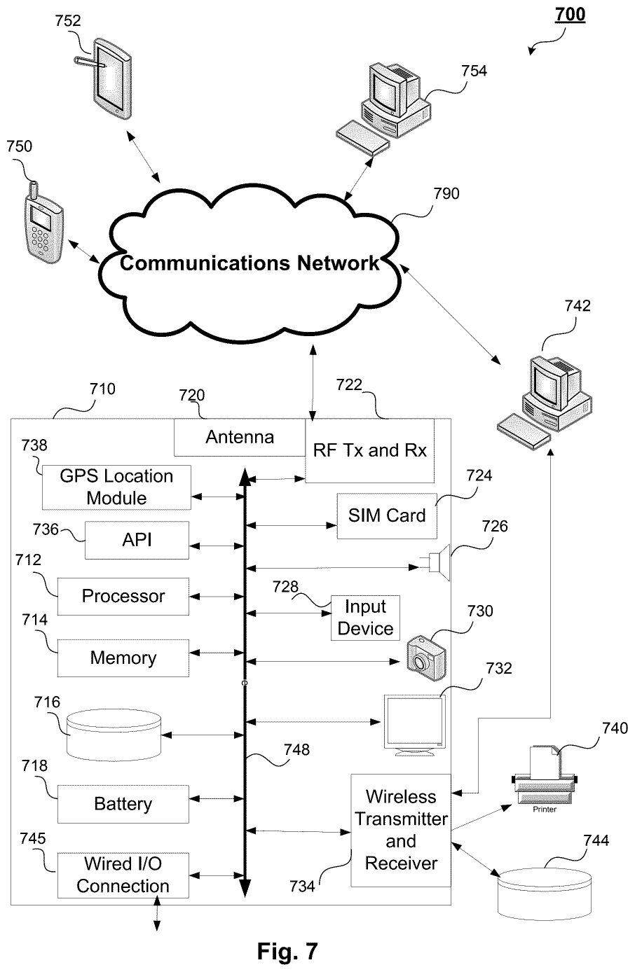

FIG. 7 is a schematic block diagram of a system 700 on which one or more aspects of brain stimulation method and system of the present disclosure may be practised. The system 700 includes a portable computing device in the form of a smartphone 710, which may be used by a registered user of the brain stimulation system in FIG. 1. The smartphone 710 includes a plurality of components, including: a processor 712, a memory 714, a storage medium 716, a battery 718, an antenna 720, a radio frequency (RF) transmitter and receiver 722, a subscriber identity module (SIM) card 724, a speaker 726, an input device 728, a camera 730, a display 732, and a wireless transmitter and receiver 734. Components of the smartphone 710 generally communicate using one or more bus connections 748 or other connections therebetween. The smartphone 710 also includes a wired connection 745 for coupling to a power outlet to recharge the battery 718 or for connection to a computing device, such as the general purpose computer 610 of FIG. 6. The wired connection 745 may include one or more connectors and may be adapted to enable uploading and downloading of content from and to the memory 714 and SIM card 724.

The smartphone 710 may include many other functional components, such as an audio digital-to-analogue and analogue-to-digital converter and an amplifier, but those components are omitted for the purpose of clarity. However, such components would be readily known and understood by a person skilled in the relevant art.

The memory 714 may include Random Access Memory (RAM), Read Only Memory (ROM), or a combination thereof. The storage medium 716 may be implemented as one or more of a solid state “flash” drive, a removable storage medium, such as a Secure Digital (SD) or microSD card, or other storage means. The storage medium 716 may be utilised to store one or more computer programs, including an operating system, software applications, and data. In one mode of operation, instructions from one or more computer programs stored in the storage medium 716 are loaded into the memory 714 via the bus 748. Instructions loaded into the memory 714 are then made available via the bus 748 or other means for execution by the processor 712 to implement a mode of operation in accordance with the executed instructions.

The smartphone 710 also includes an application programming interface (API) module 736, which enables programmers to write software applications to execute on the processor 712. Such applications include a plurality of instructions that may be pre-installed in the memory 714 or downloaded to the memory 714 from an external source, via the RF transmitter and receiver 722 operating in association with the antenna 720 or via the wired connection 745.

The smartphone 710 further includes a Global Positioning System (GPS) location module 738. The GPS location module 738 is used to determine a geographical position of the smartphone 710, based on GPS satellites, cellular telephone tower triangulation, or a combination thereof. The determined geographical position may then be made available to one or more programs or applications running on the processor 712.

The wireless transmitter and receiver 734 may be utilised to communicate wirelessly with external peripheral devices via Bluetooth, infrared, or other wireless protocol. In the example of FIG. 7, the smartphone 710 is coupled to each of a printer 740, an external storage medium 744, and a computing device 742. The computing device 742 may be implemented, for example, using the general purpose computer 610 of FIG. 6.

The camera 726 may include one or more still or video digital cameras adapted to capture and record to the memory 714 or the SIM card 724 still images or video images, or a combination thereof. The camera 726 may include a lens system, a sensor unit, and a recording medium. A user of the smartphone 710 may upload the recorded images to another computer device or peripheral device using the wireless transmitter and receiver 734, the RF transmitter and receiver 722, or the wired connection 745.

In one example, the display device 732 is implemented using a liquid crystal display (LCD) screen. The display 732 is used to display content to a user of the smartphone 710. The display 732 may optionally be implemented using a touch screen, such as a capacitive touch screen or resistive touchscreen, to enable a user to provide input to the smartphone 710.

The input device 728 may be a keyboard, a stylus, or microphone, for example, for receiving input from a user. In the case in which the input device 728 is a keyboard, the keyboard may be implemented as an arrangement of physical keys located on the smartphone 610. Alternatively, the keyboard may be a virtual keyboard displayed on the display device 732.

The SIM card 724 is utilised to store an International Mobile Subscriber Identity (IMSI) and a related key used to identify and authenticate the user on a cellular network to which the user has subscribed. The SIM card 724 is generally a removable card that can be used interchangeably on different smartphone or cellular telephone devices. The SIM card 724 can be used to store contacts associated with the user, including names and telephone numbers. The SIM card 724 can also provide storage for pictures and videos. Alternatively, contacts can be stored on the memory 714.

The RF transmitter and receiver 722, in association with the antenna 720, enable the exchange of information between the smartphone 710 and other computing devices via a communications network 790. In the example of FIG. 7, RF transmitter and receiver 722 enable the smartphone 710 to communicate via the communications network 790 with a cellular telephone handset 750, a smartphone or tablet device 752, a computing device 754 and the computing device 742. The computing devices 754 and 742 are shown as personal computers, but each may be equally be practised using a smartphone, laptop, or a tablet device, or the EEG hardware 110 of FIG. 1 used to detect signals from a patient.

The communications network 790 may be implemented using one or more wired or wireless transmission links and may include, for example, a cellular telephony network, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a cellular (mobile) telephone cellular network, a short message service (SMS) network, or any combination thereof.

When one or more functions of the brain stimulation system described herein are implemented using the smartphone 710 of FIG. 7, a software application (“app”) executing on the processor 712 may be utilised to implement any one or more of the functions described and shown in relation to FIGS. 1 to 5. In some implementations, the app is a native app executing on the smartphone 700. In alternative implementations, the app is a web-based app displayed in a browser executing on the processor 712, with the smartphone 710 coupled to a remote server, such as the computing device 742, on which the app is executing.

### INDUSTRIAL APPLICABILITY

The arrangements described are applicable to the medical and health industries, as well as having broader industrial applications, such as in education, workplace productivity, occupational safety, and defence.

The foregoing describes only some embodiments of the present invention, and modifications and/or changes can be made thereto without departing from the scope and spirit of the invention, the embodiments being illustrative and not restrictive.

Reference throughout this specification to “one embodiment”, “an embodiment,” “some embodiments”, or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

While some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practised without these specific details. In other instances, well-known methods, structures and techniques have not been shown in detail in order not to obscure an understanding of this description.

Note that when a method is described that includes several elements, e.g., several steps, no ordering of such elements, e.g., of such steps, is implied, unless specifically stated.

In the context of this specification, the word “comprising” and its associated grammatical constructions mean “including principally but not necessarily solely” or “having” or “including”, and not “consisting only of”. Variations of the word “comprising”, such as “comprise” and “comprises” have correspondingly varied meanings.

Similarly, it is to be noticed that the term coupled should not be interpreted as being limitative to direct connections only. The terms “coupled” and “connected,” along with their derivatives, may be used. It should be understood that these terms are not intended as synonyms for each other but may be. Thus, the scope of the expression “a device A coupled to a device B” should not be limited to devices or systems wherein an input or output of device A is directly connected to an output or input of device B. It means that there exists a path between device A and device B which may be a path including other devices or means in between. Furthermore, “coupled to” does not imply direction. Hence, the expression “a device A is coupled to a device B” may be synonymous with the expression “a device B is coupled to a device A”. “Coupled” may mean that two or more elements are either in direct physical or electrical contact, or that two or more elements are not in direct contact with each other but yet still co-operate or interact with each other.

As used throughout this specification, unless otherwise specified, the use of ordinal adjectives “first”, “second”, “third”, “fourth”, etc., to describe common or related objects, indicates that reference is being made to different instances of those common or related objects, and is not intended to imply that the objects so described must be provided or positioned in a given order or sequence, either temporally, spatially, in ranking, or in any other manner.

Although the invention has been described with reference to specific examples, it will be appreciated by those skilled in the art that the invention may be embodied in many other forms.

1. A brain stimulation system for use on a mammal comprising:
an electroencephalogram (EEG) headset having a plurality of EEG sensors for detecting an electrical signal of a patient, each sensor corresponding to a channel of the signal;
a computer-implemented EEG processing device for determining a stimulation protocol based on said detected electrical signal, wherein said EEG processing device includes:
a brain state inference module for determining an inferred brain state of the patient for a given time based on said detected electrical signal and a set of predefined brain states, wherein each predefined brain state is associated with a set of brain state parameters including corresponding brain network patterns and brain network dynamics, said brain state inference module determining a likelihood of the patient being in each of said predefined brain states based on: (i) correspondence of said detected electrical signal with said set of brain network patterns associated with each of said predefined brain states, and (ii) said brain network dynamics, wherein said inferred brain state has the highest likelihood for said given time, and an artificial-intelligence implemented learning module for learning said stimulation protocol based on the detected electrical signal and said inferred brain state; and
a neuromodulator for delivering stimuli to said patient based on said stimulation protocol.

2. The brain stimulation system according to claim 1, wherein said EEG processing device further includes:
a pre-processing module for pre-processing the electrical signals before processing by said brain state inference module, wherein pre-processing includes at least one of: removing artefacts, removing extraneous data, and formatting the signal.

3. The brain stimulation system according to claim 2, wherein said pre-processing module includes:
a memory buffer and resampling module for regulating transmission of samples at a sampling rate equal to or lower than the sampling rate of the original EEG acquisition;
a time domain filter for applying linear time invariant filtering to each channel of said electrical signals;
an artefact identifier that filters the electrical signal to identify and remove artefacts; and
dimensionality reduction module to reduce the inherent dimensionality of the signal.

4. The brain state-stimulation system according to claim 1, wherein the brain state inference module is implemented using a probabilistic latent state model.

5. The brain state-stimulation system according to claim 4, wherein the probabilistic latent state model is selected from the group consisting of: a Time-Delay Embedded Hidden Markov Model (HMM); state dynamics modelled using recurrent neural networks; state dynamics modelled using temporal convolutional neural networks; non-Gaussian distributions over raw data; and application of nonlinear transformations to raw data.

6. The brain stimulation system according to claim 1, wherein said learning module applies a reinforcement learning algorithm to determine said stimulation protocol based on how the patient responds to applied stimulation.

7. The brain stimulation system according to claim 1, wherein said neuromodulator is selected from the group consisting of: Closed Loop Auditory Stimulation (CLAS); AudioVisual Stimulation (AVS); Gamma Entrainment Using Sensory Stimulus Stimulation (GENUS); Transcranial Magnetic Stimulation (TMS); Transcranial Direct Current Stimulation (TDCS); Transcranial Alternating Current Stimulation (TACS); and
Transcranial Ultrasound Stimulation (TUS).

8. The brain stimulation system according to claim 1, wherein said stimulation protocol relates to turning a stimulus to one of an on or off position.

9. The brain stimulation system according to claim 1, wherein said stimulation protocol relates to at least one stimulation parameter selected from the group consisting of: timing, location, orientation, magnitude, frequency, and duration.

10. The brain stimulation system according to claim 9, wherein said stimulation protocol relates to:
controlling at least one of an audio signal, visual signal, and pattern; or
controlling the spatial position of robotically-guided TMS neuromodulation.

11. (canceled)

12. The brain stimulation system according to claim 1, further comprising:
neuromodulatory hardware that include at least one of: a TMS coil, a signal generation unit, a cooling unit, a power supply, a robotic TMS positioning arm, a robotic control unit, an optical position sensor, a camera, a pointer tool, and a coil tracker.

13. The brain stimulation system according to claim 1, wherein said learning module utilizes a reinforcement learning algorithm to determine said stimulation protocol.

14. The brain stimulation system according to claim 13, wherein the reinforcement learning algorithm is selected from the group consisting of: Q-learning; a state-action-reward-state-action (SARSA) algorithm; a temporal difference learning algorithm; an actor-critic method; a Monte Carlo reinforcement learning method; and a deep reinforcement learning method.

15. The brain stimulation system according to claim 1, wherein said EEG headset is configured such that said EEG sensors are positioned at predefined locations of the scalp of said patient when said headset is worn by said patient.

16. The brain stimulation system according to claim 1, wherein said EEG headset includes a wireless transmitter for coupling said EEG headset to said EEG processing device.

17. The brain stimulation system according to claim 16, wherein said wireless transmitter utilizes a wireless transmission protocol selected from the group consisting of: radiofrequency (RF), Bluetooth, Wi-Fi, Zigbee, Z Wave, 6LowPAN, GRPS/3G/4G/5G/LTE, and Near Field Communication (NFC).

18. The brain stimulation system according to claim 1, wherein said learning module learns the stimulation protocol through a reward function step, a learning algorithm step, and a policy implementation step.

19. (canceled)

20. A method of stimulating a brain of a mammal comprising the steps of:
utilising an electroencephalogram (EEG) headset to detect an electrical signal of a patient, said EEG headset having a plurality of EEG sensors wherein each sensor corresponds to a channel of the signal;
determining a stimulation protocol based on said detected electrical signal by utilising a computer-implemented EEG processing device, wherein said EEG processing device includes:
a storage medium for storing a set of predefined brain states, each brain state being associated with a set of brain state parameters including corresponding brain network patterns and brain network dynamics;
a brain state inference module for determining an inferred brain state of the patient for a given time based on said detected electrical signal and said set of predefined brain states, said brain state inference module determining a likelihood of the patient being in each of said predefined brain states based on: (i) correspondence of said detected electrical signal with said set of brain network patterns associated with each of said predefined brain states, and (ii) said brain network dynamics, wherein said inferred brain state has the highest likelihood for said given time, and
an artificial-intelligence implemented learning module for learning said stimulation protocol based on the detected electrical signal and said inferred brain state; and
delivering stimuli to said patient, via a neuromodulator, based on said stimulation protocol.

21. (canceled)

22. The method according to claim 20, wherein said stimulation protocol relates to at least one of magnitude, frequency, and duration for at least one stimulus parameter.

23. A brain stimulation system for use on a mammal comprising:
a neurophysiological sensing device having a plurality of sensors for detecting a neurophysiological signal of a patient, each sensor corresponding to a channel of the signal;
a computer-implemented processing device for determining a stimulation protocol based on said detected neurophysiological signal, wherein said processing device includes:
a brain state inference module for determining an inferred brain state of the patient for a given time based on said detected electrical signal and a set of predefined brain states, wherein each predefined brain state is associated with a set of brain state parameters including corresponding brain network patterns and brain network dynamics, said brain state inference module determining a likelihood of the patient being in each of said predefined brain states based on: (i) correspondence of said detected electrical signal with said set of brain network patterns associated with each of said predefined brain states, and (ii) said brain network dynamics, wherein said inferred brain state has the highest likelihood for said given time, and
an artificial-intelligence implemented learning module for learning said stimulation protocol based on the detected electrical signal and said inferred brain state; and
a neuromodulator for delivering stimuli to said patient based on said stimulation protocol.
